(19) United States
(12) Patent Application Publication
(10) Pub. No.: US 2002/0187249 A1
(43) Pub. Date: Dec. 12, 2002

(54) Process for the production of surface-functionalized supports that serve as starting materials for microarrays used for immobilizing biomolecules

(76) Inventors: Wilhelm Pluster, Boulder, CO (US); Mathias Ulbricht, Berlin (DE); Heinz Gerhard Kohn, Hamburg (DE)

Correspondence Address: DAVID TOREN, ESQ., SIDLEY, AUSTIN, BROWN & WOOD, LLP, 787 SEVENTH AVENUE, NEW YORK, NY, 10019-6018, US

(21) Appl. No.: 10/062,024

(22) Filed: Feb. 1, 2002

(30) Foreign Application Priority Data
May 8, 2001 (EP) PCT/EP01/05173

(51) Int. Cl. A61L 2/00

(52) U.S. Cl. 427/2.1

(57) ABSTRACT

A process for the production of surface-functionalized supports that serve as starting products for the production of microarrays for immobilizing biomolecules, in which process the surface of a support is coated with an initiator and the coated surface is then put in contact with a solution containing at least one first group of polymerizable monomers, whereby the monomers contain binding sites onto which the biomolecules (probe molecules) can bond, and whereby the conditions under which the monomers are put in contact with the activated support are selected in such a way that the monomers, mediated by the initiator, bind to the support and, on that basis, polymerize to form functional polymer chains in such a manner that a fixed structure consisting of adjacent functional polymer chains is formed on the surface of the support.

### BACKGROUND

The invention relates to a process for the production of surface-functionalized supports for immobilizing biomolecules that are to be used in the production of microarrays, it relates to a process for the production of such microarrays starting with the surface-functionalized supports produced according to the invention and it also relates to the supports and microarrays that can be produced with the process.

The term microarrays as used herein refers to flat supports (slides) on whose surface biomolecules that serve as probes (probe molecules) are coupled in defined areas (spots) isolated from each other, said biomolecules, in turn, being capable of establishing a specific bond to other biomolecules that are to be analyzed (target molecules). These spots are detected as two-dimensional objects. The term array should be interpreted broadly and, in the sense used here, also encompasses other distributions of spots on the support surface which do not necessarily fall within a strict definition of an array arrangement.

The term support as used below refers to the untreated slides made of glass or plastic. After functionalization, the supports are designated as functionalized supports, whereby no probe molecules are coupled here yet. The term microarrays is only employed for functionalized supports to which probe molecules are coupled.

Microarrays are especially used, for example, to analyze complex mixtures containing DNA or RNA or for protein analysis. The technique that employs microarrays will also be referred to below as array technique.

Oligonucleotides, for instance, are employed as probe molecules for DNA and RNA analyses. For protein analyses, antigens or antibodies can be coupled to the supports as probe molecules. In particular, the invention refers to a process for the production of microchips for nucleic acid analysis.

The probe molecules can be synthesized, for example, directly onto the surface of the supports. Preferably, however, the probe molecules are deposited by means of a technique known as spotting onto the support surface which then has to be functionalized in such a way that it couples the probe molecules.

For the spotting procedure, that is to say, the deposition of drops containing dissolved probe molecules onto the support surface, for example, automated pinhead or ink-jet printing processes are employed which create spots containing probe molecules, preferably in an array arrangement on the support. With the conventional spotting method, the droplets normally have a size between 0.03 nL to 2 nL. Therefore, the drops have a three-dimensional nature.

The immobilization of the probe molecules by means of spotting presupposes a corresponding functionalization of the support surface. In this context, it is a known procedure to coat the support with polylysine or to carry out a silanization of the support. Both methods entail disadvantages. For instance, polylysine coating gives rise to non-specific bonds and to an undesired running of the drops after the spotting. The silanization of glass slides can also create non-specific signals that make the interpretation of the results more difficult.

U.S. Pat. No. 5,858,653 describes supports for the array technique whose surfaces have coupled hydrophilic polymers containing reactive groups which, under thermal activation, can establish a covalent bond, for example, with oligonucleotides. The supports are produced by coupling the previously synthesized polymers onto the support surface, optionally employing photoreactive groups in the polymer. A drawback here is that the relatively large polymers can only be coupled onto the support at a low loading density and with a relatively irregular distribution.

The objective of the invention is to create a process for the production of surface-functionalized supports and microarrays for use in array technology which overcomes the disadvantages of the state of the art.

### SUMMARY

The process according to the invention as set forth in claim 1 relates to the production of surface-functionalized supports which can then be further developed according to claim 16 to form microarrays, particularly microchips for nucleic acid analysis.

According to the invention, claim 1 provides that the surface of a support is first coated with an initiator which is, for example, adsorbed on—or else optionally covalently bound to—the surface of the support.

Then, the surface coated with the initiator is put in contact with a solution containing at least one first group of polymerizable monomers. The conditions under which the monomers are put in contact with the activated support surface as well as the initiator used for the activation are selected in such a way that the monomers, mediated by the initiator, bind to the support surface and, on that basis, polymerize to form functional polymer chains.

It is also provided according to the invention that the employed monomers of the first group contain binding sites onto which the biomolecules employed as probe molecules can bind, optionally with the formation of a covalent bond.

WO 00/12575 discloses polymeric solid-phase supports that are produced by means of a similar method and that particularly meet the special requirements of spotting technology. However, WO 00/12575 does not indicate that the functionalized supports described there can be further processed to form microarrays, especially microchips for nucleic acid analysis. The application purposes described in WO 00/12575 are essentially limited to the production of chemical substance libraries.

In an embodiment according to the invention, it can be provided that at least an additional second group of monomers is copolymerized with the first group on the support. The monomers of the second group essentially have no binding sites for biomolecules and serve, for example, to distribute and control the loading density of the support with binding sites and to adjust the hydrophobia or hydrophilia of the functional polymer chains.

A third group of monomers can likewise be employed which has non-covalent binding sites that are capable of directing probe molecules to the covalent binding sites. Suitable examples here are monomers having cationic groups such as, for instance, the NR₄⁺ group that is particularly suitable for nucleic acids, or else anionic or chelating groups that are suitable for proteins.

The functional polymers can be formed either by polymerization of the monomers of the first group or by means of copolymerization of the monomers of the first and second groups, of the first and third groups or of the first, second and third groups.

The invention offers a large number of advantages. One essential advantage is that the functional polymer chains that have the binding sites can be formed in-situ starting with the support surface. Therefore, the support surface first reacts with monomers which, owing to their small size, can be coupled onto the support surface with a well controlled and, if so desired, relatively high density. Thus, according to the invention, the support surface can be considerably more densely loaded with the functional polymer chains formed in-situ than is possible with the known coupling of previously synthesized polymers onto the support surface.

Furthermore, the process according to the invention is particularly effective in allowing a three-dimensional distribution of the binding sites over the support surface. This increases the binding capacity for probe molecules per unit of surface area and reduces non-specific bonds, as a result of which a better signal-to-noise ratio is attained.

Another advantage is that the functional polymer chains—which are arranged next to each other by means of the process according to the invention at a density that can be well controlled—prevent rapid evaporation and running of liquid, which leads to a better yield when probe molecules are bound in a spot, to an improved uniformity over the spot surface and to a reduction of cross contamination between adjacent spots. The adjustable three-dimensional structure of the functional polymer chains ensures a stabilization of the drops deposited by the spotting procedure. In contrast to the state of the art, the deposited drops essentially maintain their three-dimensional structure when they are deposited onto the supports produced according to the invention, which accounts for more sharply defined spots.

Supports made of glass or plastic (slides) can be employed as supports according to the invention. The plastic supports can be made, for instance, of polystyrene, polycarbonate, polyvinyl chloride or else polypropylene, to name but a few examples.

As a rule, the supports have a flat shape. In a preferred embodiment of the invention, it can be provided that depressions are created in the surface area of the support intended for the functionalization. An enlargement of the support surface that can be functionalized or loaded can be easily achieved in this manner.

The depressions can have difference shapes, for instance, in the form of grooves or embossing of any kind, and they can be distributed over the support surface in a regular or irregular pattern. Preferably, it is provided that the shape and size of the depressions match each other and also that the depressions are positioned equidistantly from each other in the support surface. This gives rise to a regular pattern that can be advantageously and reproducibly loaded by commonly employed spotting equipment generating a pattern.

Particular preference is given to depressions having the shape of pyramids or cones whose tips point towards the support. Especially with depressions having this shape, the surface area of the support can be enlarged in an optimal manner. It can be assumed that the conical depression has a surface area that is 2 to 3 times larger than the cone base surface area (that is to say, a comparable surface area on a planar support), which ultimately means that the support can be functionalized at a higher density and thus be loaded with more probe molecules.

Depending on the selected type of coating with initiators, it can be necessary to additionally treat the supports in a preceding step in order, for example, to allow a coupling of the initiator onto their surfaces in the first place. A commonly employed method for supports made of glass is, for instance, silanization. Since this is also part of the state of the art, it will not be elaborated upon here. In the case of supports made of plastic, such a treatment in a preceding step is not needed as a rule.

Examples of suitable initiators are compounds that can be activated thermally, photo-chemically or through redox, such as benzoin derivatives, azo compounds or peroxides. Benzophenone is an example of a particularly well-suited initiator. Benzophenone is an initiator that can be activated by means of UV light. In order to coat the support with benzophenone, it is sufficient for the support to be immersed into a solution containing benzophenone for a certain period of time.

In a subsequent step, the support coated, for example, with benzophenone, is equilibrated with the monomers. In the case of benzophenone activation, this is then followed by irradiation with UV light, whereby the benzophenone mediates a binding of the monomers onto the support surface.

The described in-situ technique with the mediation of an initiator that can be induced by UV light is referred to as photo-initiated graft polymerization and is described, for example, in the article by Ulbrich et al. in Colloids and Surfaces, volume 138, 1988, page 353. The substrates that can be produced by means of graft polymerization described there are supposed to be utilized especially for ultrafiltration purposes. Neither utilization in array technology nor the specific production for this technology can be derived from that publication.

Naturally, other initiators are also conceivable which, by virtue of their special configuration or reactivity, can mediate a bond between the support surface and the monomer. It is also conceivable here for the initiator to be incorporated into the molecule formed or else, for example, like in the case of benzophenone, for the initiator to only mediate the bond but not be incorporated itself.

Examples of suitable monomers are all compounds that can be polymerized well, such as acrylic acid, methacrylic acid, derivatives of acrylic acid or methacrylic acid as well as vinyl and allyl compounds.

Particularly well-suited monomers of the first group that additionally have binding sites that are capable of binding, optionally covalently binding, the desired probe molecules are acrylic acid, glycidyl methacrylate or aminoalkyl methacrylate.

Suitable monomers of the second group are all compounds whose properties allow the hydrophilia or hydrophobia of the functional polymer chains to be adjusted. Polyethylene glycol, methacrylates or hydroxymethyl methacrylamide, for instance, are especially well-suited.

As a rule, the binding sites are functional groups that are contained in the monomers. In particular, such binding sites are COOH—, SH—, NH₂—, epoxide or thiol groups which allow a covalent binding of the probe molecule. The monomers or functional groups that serve as binding sites can be easily selected so as to be adapted to the probe molecules to be bonded. This does not pose a problem for the person skilled in the art.

The surface-functionalized supports according to the invention are to be employed primarily in defined spots as microarrays after they have been coupled with probe molecules. The array technology is a common method, particularly in conjunction with nucleic acid analyses, as a result of which this will not be elaborated upon here.

An essential feature of this technique is the use of microarrays to which probe molecules are coupled at defined sites, said molecules having highly specific binding properties, for example, for nucleic acid molecules with certain base sequences or proteins with certain immunological properties.

As a rule, the defined sites are loaded with such probe molecules automatically, as mentioned above, by means of a technique known as spotting. Spotting is a familiar technique which will not be elaborated upon here.

The surface-functionalized supports produced according to the invention can be loaded with probe molecules, for example, by means of spotting techniques, and can then be further processed to form microarrays.

In this context, for instance, plasmid DNA, cosmid DNA, bacteriophage DNA, genomic DNA, RNA, cDNA, pDNA and oligonucleotides, to name but a few examples, can be coupled. Naturally, it is also conceivable that antigens or antibodies for protein analysis as well as other biomolecules can also be coupled to the above-mentioned binding sites.

The invention is not limited to a process for the production of surface-functionalized supports or microarrays only, but rather, it also encompasses supports and arrays that can be produced by means of this process.

In particular, the invention is meant to encompass processes for the production of surface-functionalized supports that can be further processed to make microchips for nucleic acid analysis and it also encompasses such microchips.

The invention will be explained in greater detail below with reference to an example and three figures.

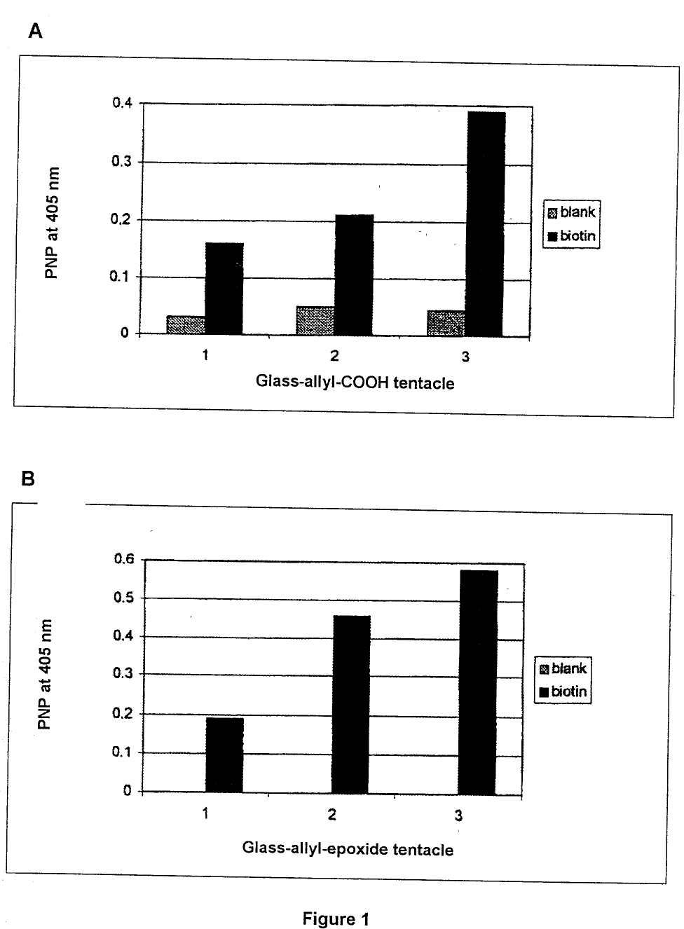

FIG. 1—documents the binding behavior of an embodiment of the functionalized support that can be produced according to the invention,

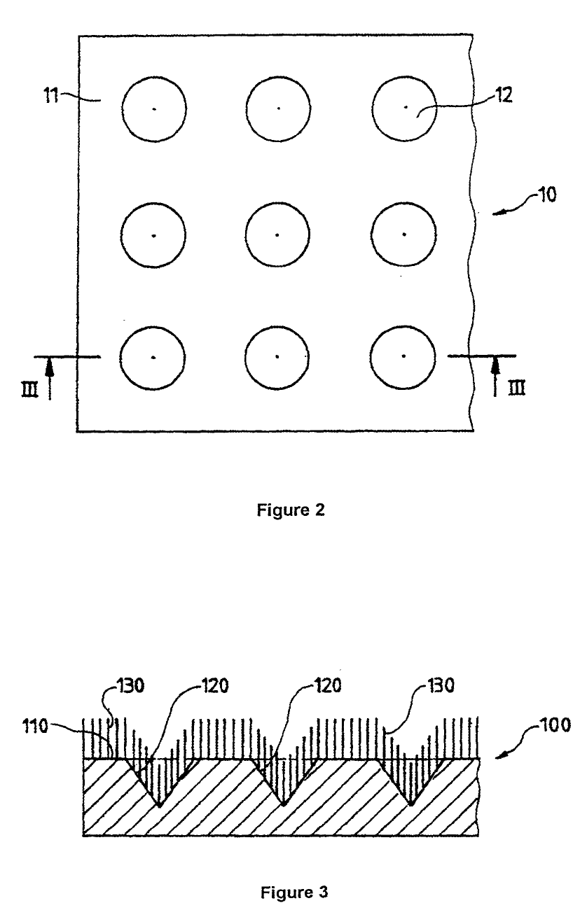

FIG. 2—shows a top view of an embodiment of the support that can be used according to the invention,

FIG. 3—shows a cross section of the surface-functionalized support produced starting with the support from FIG. 2.

### DETAILED DESCRIPTION OF THE PREFERRED EMBODIMENT

### EXAMPLE

Production of Functionalized Glass Supports

Glass supports (CCT, Jena) that were silanized on one side were employed. The glass supports were immersed into a 100 mM-solution of benzophenone in acetone for 15 minutes, subsequently rinsed with an mM-solution of benzophenone in acetone and finally dried in the air. Then the glass supports were placed with their side that was silanized and coated with benzophenone onto the monomer solutions described below with the addition of 1 mM of sodium periodate without the inclusion of air bubbles between the glass and the solution. After an equilibration time of 15 minutes, the specimens floating on the monomer solution were exposed to light through the glass. After another reaction time lasting 15 minutes, the supports were thoroughly washed with water, then with acetone and subsequently with water once again. Finally, the specimens were dried.

A) Acrylic acid at a concentration of 25 g/L of water was used as the monomer solution. This monomer solution was treated for different exposure times (t_{UV})

The binding sites prepared with these monomers were carboxyl groups.

The differing variants can be summarized as follows:

| | |
|---|---|
| 1) acrylic acid 25 g/L | 7.5 min t_{UV} |
| 2) acrylic acid 25 g/L | 10 min t_{UV} |
| 3) acrylic acid 25 g/L | 15 min t_{UV} |

B) Glycidyl methacrylate at a concentration of 10 g/L of water and 20 g/L of water, each with 25 g/L of hydroxymethyl methacrylamide, was used as the monomer solution. Here, too, different exposure times t_{UV} of 10 minutes (10 and 20 g/L of water) or 15 minutes (only with 20 g/L of water) were employed.

The binding sites prepared with these monomers were epoxide groups.

The differing variants can be summarized as follows:

| | |
|---|---|
| 1) glycidyl methacrylate 10 g/L | 10 min t_{UV} |
| 2) glycidyl methacrylate 20 g/L | 10 min t_{UV} |
| 3) glycidyl methacrylate 20 g/L | 15 min t_{UV} |

+25 g/L of hydroxymethyl methacrylamide in each case.

The specimen underwent biomolecule immobilization and biomolecule assay.

A) Biotin was coupled via N-aminoethyl biotinamide (“Biotinamin”; Molecular Probes) to the surfaces activated with EDC/NHS for 1 hour at 25° C. [77° F.] (reaction at a pH value of 7.4 for 5 hours at 25° C. [77° F.]); blank specimens were prepared analogously but without “Biotinamin”.

B) Biotin was coupled to the surfaces via “Biotinamin” (reaction at a pH value of 9.6 over-night at 25° C. [77° F.]); blank specimens were obtained through incubation overnight at 25° C. [77° F.] in a buffer at a pH value of 9.6.

The biotin assay for A and B was carried out in each case with 16 mm² of functionalized glass: 1) incubation with a streptavidin-alkaline phosphatase conjugate for 30 minutes at room temperature (RT); 2) reaction with paranitrophenyl phosphate (PNP) for 30 minutes at 37° C. [98.6° F.] and photometric measurement of the conversion at 405 nm.

The results of the photometric measurement are shown in FIG. 1.

It can be seen in both cases that there is a very efficient binding of biotin to the functionalized supports. The background signal (here through non-specific binding of the streptavidin conjugate) can be minimized by the appropriate selection of the monomers (B) or by regulating the loading density (A2 and A3).

FIG. 2 shows a flat support 10 having a surface area 11 intended for the functionalization and in this area, there are conical depressions 12 provided at regular intervals. The conical depressions can have a diameter, for instance, of approximately 20 μm and are at a distance from each other of likewise 20 μm. The usual spots have a diameter of about 100 μm to 150 μm, thus covering between 20 and 40 conical depressions on the surface 11 of the support 10. Naturally, these figures are not mandatory. It is equally possible to employ supports whose surfaces have depressions with a different shape, diameter, etc. The essential aspect, however, is that, whenever possible, the depressions should taper towards the interior of the support since the slanted faces thus created bring about the desired surface area enlargement.

The support 10 shown in FIG. 2 has not yet been surface-functionalized. The surface-functionalized state is depicted in FIG. 3 in a sectional view. Here, one can see a support 100 having a functionalized surface 110 which has been provided with depressions 120. The polymer chains covalently coupled to the surface 110 within the scope of the functionalization have been designated with the reference numeral 130. It is evident that more polymer chains 130 can be coupled per conical depression 120 than would be possible on a comparable planer section of the surface 110, whose size corresponds to that of the cone base surface.

1. A process for the production of surface-functionalized supports that serve as starting products for the production of microarrays for immobilizing biomolecules, in which process the surface of a support is coated with an initiator and the coated surface is then put in contact with a solution containing at least one first group of polymerizable monomers, whereby the monomers contain binding sites onto which the biomolecules (probe molecules) can bind, and whereby the conditions under which the monomers are put in contact with the activated support are selected in such a way that the monomers, mediated by the initiator, bind to the support and, on that basis, polymerize to generate functional polymer chains in such a manner that a fixed structure consisting of adjacent functional polymer chains is formed on the surface of the support.

2. The process according to claim 1, wherein substances that can be activated by light, especially benzophenone, are employed as the initiator.

3. The process according to claim 1 wherein the supports are made of glass or plastic.

4. The process according to claim 3, wherein the plastic supports employed consist of polystyrene, polycarbonate, polyvinyl chloride or polypropylene.

5. The process according to claim 1, wherein the supports are configured so as to be flat and they have depressions in a surface area intended for the functionalization.

6. The process according to claim 5, wherein the support surface is provided with depressions whose shapes and sizes match each other and which are positioned equidistantly from each other.

7. The process according to claim 5, wherein the depressions have the shape of pyramids or cones whose tips point towards the support.

8. The process according to one of claim 1, wherein the supports made of glass employed in the process are silanized in a first step.

9. The process according to one of claim 1, wherein in that at least an additional second group of monomers is copolymerized with the first group on the support, whereby the monomers of the second group essentially do not have any binding sites for biomolecules.

10. The process according to claim 9, wherein the selection of the monomers of the second group and the determination of the monomer concentration to be employed are both carried out with an eye towards the adjustment of the desired hydrophilia or hydrophobia of the copolymerized functional polymer chains.

11. The process according to claim 10, wherein the selection of the monomers of the second group and the concentration employed lead to copolymerized functional polymer chains that receive and retain the three-dimensional structure of a drop deposited during spotting.

12. The process according to claim 1, wherein that the monomers of the first and second groups are selected from among the following compounds: acrylic acids, methacrylic acids, derivatives of acrylic acid or methacrylic acid as well as vinyl or allyl compounds.

13. The process according to claim 12, wherein the monomers of the first group are selected from among the following compounds: acrylic acids, glycidyl methacrylates or aminoalkyl methacrylates.

14. The process according to claim 13, wherein the monomers of the second group are selected from among the following compounds: polyethylene glycols, methacrylates or hydroxymethyl methacrylamides.

15. The process according to claim 1, wherein the binding sites contained in the monomers of the first group are functional groups, particularly COOH—, SH—, NH₂—, epoxide or thiol groups.

16. A process for the production of microarrays wherein probe molecules dissolved in a solvent are deposited dropwise in defined areas on a surface-modified support produced according to claim 1.

17. The process for the production of microarrays according to claim 16, wherein the surface-modified supports are loaded with probe molecules by means of spotting.

18. The process according to claim 16, wherein oligonucleotides or antibodies are employed as the probe molecules.

19. The process for the production of microarrays according to claim 16, wherein the microarrays are made as microchips for nucleic acid analysis.

20. The surface-modified supports that can be produced according to claim 1.

21. A support for the array binding technique that can be produced according to claim 16.
